(19) United States
(12) Patent Application Publication
(10) Pub. No.: US 2024/0136024 A1
(43) Pub. Date: Apr. 25, 2024

(54) APPARATUS AND METHOD WITH MOLECULAR DYNAMICS SIMULATION

(71) Applicant: SAMSUNG ELECTRONICS CO., LTD., Suwon-si (KR)

(72) Inventors: Lun YAO, Xi'an (CN); Jiali PANG, Xi’an (CN); Ihor VASYLTSOV, Suwon-si (KR); Gang SUN, Xi’an (CN); Zhen ZHANG, Xi’an (CN)

(73) Assignee: SAMSUNG ELECTRONICS CO., LTD., Suwon-si (KR)

(21) Appl. No.: 18/480,190

(22) Filed: Oct. 3, 2023

(30) Foreign Application Priority Data
Oct. 12, 2022 (CN) 202211248999.2
Jun. 29, 2023 (KR) 10-2023-0084257

(51) Int. Cl. G16C 10/00

(52) U.S. Cl. CPC G16C 10/00

(57) ABSTRACT

A processor-implemented method with molecular dynamics simulation includes: setting a precision of first data used for a molecular dynamics simulation to be a first precision; setting a precision of second data used for the molecular dynamics simulation to be a second precision that is different from the first precision; and conducting the molecular dynamics simulation based on the first data of the first precision and the second data of the second precision.

### CROSS-REFERENCE TO RELATED APPLICATIONS

This application claims the benefit under 35 USC § 119(a) of Chinese Patent Application No. 202211248999.2 filed on Oct. 12, 2022, in the China National Intellectual Property Administration, and Korean Patent Application No. 10-2023-0084257 filed on Jun. 29, 2023, in the Korean Intellectual Property Office, the entire disclosures of which are incorporated herein by reference for all purposes.

### BACKGROUND

### 1. Field

The following description relates to a method and apparatus with molecular dynamics simulation.

### 2. Description of Related Art

Molecular dynamics simulation may calculate a characteristic and a structure of a system by simulating a motion of an atom in a system constituted by the atom using a computer and may be an important simulation method for evaluating and predicting a structure and characteristic of a substance from a microscopic aspect of the substance.

In an actual molecular dynamics simulation calculation process, to obtain ideal experimental data, it may need to expand the scale of the simulation calculation and extend the simulation time, and thus, the simulation calculation may consume considerable time. A temporal step of molecular dynamics simulation may be a few femtoseconds, whereas a computer simulation with millions of steps may correspond to a few femtoseconds in an actual process and the computational amount may be significantly large to reflect an actual macroscopic operation.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one or more general aspects, a processor-implemented method with molecular dynamics simulation includes: setting a precision of first data used for a molecular dynamics simulation to be a first precision; setting a precision of second data used for the molecular dynamics simulation to be a second precision that is different from the first precision; and conducting the molecular dynamics simulation based on the first data of the first precision and the second data of the second precision.

The first data may include either one or both of position coordinates and force coordinates of an atom, the second data may include either one or both of a potential energy parameter and velocity coordinates of the atom, and the second precision may include precision that is higher than the first precision.

The method may include: receiving input data comprising initial position coordinates of an atom and an interatomic initial interaction potential; and segmenting an initial atom space corresponding to the initial position coordinates into a plurality of subdomains processed by a plurality of processes in a distributed environment, wherein the conducting of the molecular dynamics simulation may include iteratively updating position coordinates, force coordinates, and velocity coordinates of each atom comprised in each of the plurality of subdomains through the plurality of processes based on the input data.

The plurality of subdomains may include a first subdomain and a second subdomain neighboring to the first subdomain, the plurality of processes may include a first process corresponding to the first subdomain and a second process corresponding to the second subdomain, and the iteratively updating may include: updating force coordinates of an atom of the first subdomain and force coordinates of an atom of the second subdomain; parallelly quantify the force coordinates of the atom of the first subdomain and the force coordinates of the atom of the second subdomain; synchronizing the force coordinates of the atom of the first subdomain with the force coordinates of the atom of the second subdomain; and dequantizing synchronized force coordinates of the atom of the first subdomain and synchronized force coordinates of the atom of the second subdomain.

The iteratively updating may include iteratively updating each of the plurality of subdomains.

The iteratively updating of each of the plurality of subdomains may include: determining position coordinates of the atom of a current update based on position coordinates of the atom of a previous update and velocity coordinates of the atom of the previous update; determining force coordinates of the atom based on the position coordinates of the atom in the current update and the force coordinates of the atom of the current update; and determining velocity coordinates of the atom of the current update based on velocity coordinates of the atom of a previous update and velocity coordinates of the atom of the previous update.

The determining of the position coordinates of the atom may include determining position coordinates of the atom corresponding to a first update to be the initial position coordinates, and

the determining of the velocity coordinates of the atom of the current update may include determining the velocity coordinates of the atom through the interatomic initial interaction potential, wherein the velocity coordinates of the atom corresponds to the first update.

The determining of the force coordinates of the atom of the current update may include:

determining the interatomic interaction potential based on the position coordinates of the atom of the current update; and determining the force coordinates of the atom of the current update based on the interatomic interaction potential.

For each atom, the determining of the force coordinates of the atom of the current update based on the interatomic interaction potential may include: determining a plurality of force coordinates applied to the atom by another atom based on the interatomic interaction potential; determining sum force coordinates of the plurality of force coordinates; and converting the sum force coordinates into force coordinates of the atom of the current update.

Precision of the plurality of force coordinates and precision of the force coordinates of the atom of the current update may be the first precision, precision of the sum force coordinates may be the second precision, and the second precision may include precision that is higher than the first precision.

In one or more general aspects, an apparatus with molecular dynamics simulation includes: one or more processors configured to: set a precision of first data used for a molecular dynamics simulation to be first precision; set a precision of second data used for the molecular dynamics simulation to be second precision that is different from the first precision; and conduct the molecular dynamics simulation based on the first data of the first precision and the second data of the second precision.

The first data may include either one or both of position coordinates and force coordinates of an atom, the second data may include either one or both of a potential energy parameter and velocity coordinates of the atom, and the second precision may include precision that is higher than the first precision.

The one or more processors may be configured to: receive input data comprising initial position coordinates of an atom and an interatomic initial interaction potential; segment an initial atom space corresponding to initial position coordinates into a plurality of subdomains processed by a plurality of processes in a distributed environment; and for the conducting of the molecular dynamics simulation, iteratively update position coordinates, force coordinates, and velocity coordinates of each atom comprised in each of the plurality of subdomains through the plurality of processes based on the input data.

The plurality of subdomains may include a first subdomain and a second subdomain neighboring to the first subdomain, the plurality of processes may include a first process corresponding to the first subdomain and a second process corresponding to the second subdomain, and for the iteratively updating, the one or more processors may be configured to: update the force coordinates of the atom of the first subdomain with the force coordinates of the atom of the second subdomain; parallelly quantify the force coordinates of the atom of the first subdomain and the force coordinates of the atom of the second subdomain; synchronize the force coordinates of the atom of the first subdomain with the force coordinates of the atom of the second subdomain; and dequantize synchronized force coordinates of the atom of the first subdomain and synchronized force coordinates of the atom of the second subdomain.

For the iteratively updating, the one or more processors may be configured to iteratively update each of the plurality of subdomains.

For the iteratively updating of each of the plurality of subdomains, the one or more processors may be configured to: determine position coordinates of the atom of a current update based on position coordinates of the atom of a previous update and velocity coordinates of the atom of the previous update; determine force coordinates of the atom based on the position coordinates of the atom in the current update and the force coordinates of the atom of the current update; and determine velocity coordinates of the atom of the current update based on velocity coordinates of the atom of a previous update and velocity coordinates of the atom of the previous update.

The one or more processors may be configured to: for the determining of the position coordinates of the atom, determine position coordinates of the atom corresponding to a first update to be the initial position coordinates, and for the determining of the velocity coordinates of the atom of the current update, determine the velocity coordinates of the atom through the interatomic initial interaction potential, wherein the velocity coordinates of the atom corresponds to the first update.

For the determining of the force coordinates of the atom of the current update, the one or more processors may be configured to: determine the interatomic interaction potential based on the position coordinates of the atom of the current update; and determine the force coordinates of the atom of the current update based on the interatomic interaction potential.

For the determining of the force coordinates of the atom of the current update based on the interatomic interaction potential, for each atom, the one or more processors may be configured to: determine a plurality of force coordinates applied to the atom by another atom based on the interatomic interaction potential; determine sum force coordinates of the plurality of force coordinates; and convert the sum force coordinates into force coordinates of the atom of the current update.

Precision of the plurality of force coordinates and precision of the force coordinates of the atom of the current update may be the first precision, precision of the sum force coordinates may be the second precision, and the second precision may include precision that is higher than the first precision.

Other features and aspects will be apparent from the following detailed description, the drawings, and the claims.

### BRIEF DESCRIPTION OF THE DRAWINGS

FIG. 1 illustrates an example of a schematic process of a molecular dynamics simulation method according to one or more embodiments.

FIG. 2 illustrates an example of a molecular dynamics simulation method according to one or more embodiments.

FIGS. 3A and 3B illustrate examples of a subdomain according to one or more embodiments.

FIG. 4 illustrates an example of a portion of a process of performing an iterative update according to one or more embodiments.

FIG. 5 illustrates an example of a portion of a process of performing an iterative update according to one or more embodiments.

FIG. 6 illustrates an example of a parallel quantization method according to one or more embodiments.

FIG. 7 illustrates an example of transmitting data through parallel quantization according to one or more embodiments.

FIG. 8 illustrates an example of parallel quantization between neighboring processes according to one or more embodiments.

FIG. 9 illustrates an example of a portion of a process of performing an iterative update according to one or more embodiments.

FIG. 10 illustrates an example of synchronizing position coordinates between neighboring processes according to one or more embodiments.

FIG. 11 illustrates an example of a schematic configuration of a molecular dynamics simulation apparatus according to one or more embodiments.

FIG. 12 illustrates an example of a detailed process of a molecular dynamics simulation method according to one or more embodiments.

FIG. 13 illustrates an example of a configuration of a molecular dynamics simulation apparatus according to one or more embodiments.

FIG. 14 illustrates an example of a molecular dynamics simulation system according to one or more embodiments.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences of operations described herein are merely examples, and are not limited to those set forth herein, but may be changed as will be apparent after an understanding of the disclosure of this application, with the exception of operations necessarily occurring in a certain order. Also, descriptions of features that are known after an understanding of the disclosure of this application may be omitted for increased clarity and conciseness.

Hereinafter, embodiments will be described in detail with reference to the accompanying drawings. However, various alterations and modifications may be made to the embodiments. Here, the embodiments are not meant to be limited by the descriptions of the present disclosure. The embodiments should be understood to include all changes, equivalents, and replacements within the idea and the technical scope of the disclosure.

The terminology used herein is for describing various examples only and is not to be used to limit the disclosure. The articles “a,” “an,” and “the” are intended to include the plural forms as well, unless the context clearly indicates otherwise. As used herein, the term “and/or” includes any one and any combination of any two or more of the associated listed items. As non-limiting examples, terms “comprise” or “comprises,” “include” or “includes,” and “have” or “has” specify the presence of stated features, numbers, operations, members, elements, and/or combinations thereof, but do not preclude the presence or addition of one or more other features, numbers, operations, members, elements, and/or combinations thereof.

Unless otherwise defined, all terms including technical and scientific terms used herein have the same meaning as commonly understood by one of ordinary skill in the art to which this disclosure pertains and based on an understanding of the disclosure of the present application. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

When describing the embodiments with reference to the accompanying drawings, like reference numerals refer to like constituent elements and a repeated description related thereto will be omitted. In the description of embodiments, detailed description of well-known related structures or functions will be omitted when it is deemed that such description will cause ambiguous interpretation of the present disclosure.

Although terms such as “first,” “second,” and “third”, or A, B, (a), (b), and the like may be used herein to describe various members, components, regions, layers, or sections, these members, components, regions, layers, or sections are not to be limited by these terms. Each of these terminologies is not used to define an essence, order, or sequence of corresponding members, components, regions, layers, or sections, for example, but used merely to distinguish the corresponding members, components, regions, layers, or sections from other members, components, regions, layers, or sections. Thus, a first member, component, region, layer, or section referred to in the examples described herein may also be referred to as a second member, component, region, layer, or section without departing from the teachings of the examples.

Throughout the specification, when a component or element is described as being “connected to,” “coupled to,” or “joined to” another component or element, it may be directly (e.g., in contact with the other component or element) “connected to,” “coupled to,” or “joined to” the other component or element, or there may reasonably be one or more other components or elements intervening therebetween. When a component or element is described as being “directly connected to,” “directly coupled to,” or “directly joined to” another component or element, there can be no other elements intervening therebetween. Likewise, expressions, for example, “between” and “immediately between” and “adjacent to” and “immediately adjacent to” may also be construed as described in the foregoing.

The same name may be used to describe an element included in the example embodiments described above and an element having a common function. Unless otherwise mentioned, the descriptions on the example embodiments may be applicable to the following example embodiments and thus, duplicated descriptions will be omitted for conciseness.

The phrases “at least one of A, B, and C”, “at least one of A, B, or C”, and the like are intended to have disjunctive meanings, and these phrases “at least one of A, B, and C”, “at least one of A, B, or C”, and the like also include examples where there may be one or more of each of A, B, and/or C (e.g., any combination of one or more of each of A, B, and C), unless the corresponding description and embodiment necessitates such listings (e.g., “at least one of A, B, and C”) to be interpreted to have a conjunctive meaning. The use of the term “may” herein with respect to an example or embodiment, e.g., as to what an example or embodiment may include or implement, means that at least one example or embodiment exists where such a feature is included or implemented, while all examples are not limited thereto.

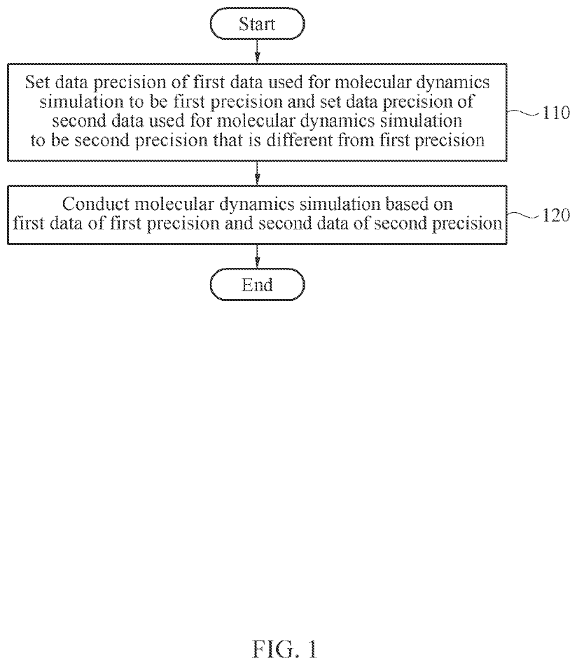

Hereinafter, a detailed description of examples of an apparatus and method of molecular dynamics simulation is provided with reference to FIGS. 1 to 14.

An example of molecular dynamics simulation is described first to help understand the present disclosure.

The molecular dynamics simulation may aim to simulate a motion state of an atom or a particle mainly in a three-dimensional (3D) space. Hereinafter, the description mainly focuses on the simulation of a motion of an atom. However, a target of simulating a motion is not limited to an atom and may be a molecule or an arbitrary particle. For example, herein, the atom may refer to an atom, a molecule, or an arbitrary particle.

In the molecular dynamics simulation, each atom may have the following three properties: position coordinates X=(x₀, x₁, x₂), force coordinates F=(f₀, f₁, f₂), and velocity coordinates V=(v₀, v₁, v₂).

In molecular dynamics, the force coordinates F (e.g., an interaction force F) between atoms may be calculated (e.g., determined) by using an interaction potential between different atoms. In addition, the velocity coordinates V of each atom may be updated by the interaction force F. Further, the position coordinates X of each atom may be updated by the velocity coordinates V. For example, the data used for molecular dynamics simulation may include position coordinates, force coordinates, velocity coordinates, and an energy parameter (e.g., an interatomic interaction potential).

FIG. 1 illustrates an example of a schematic process of a molecular dynamics simulation method according to one or more embodiments.

A molecular dynamics simulation method of one or more embodiments may improve the efficiency of molecular dynamics simulation. Referring to FIG. 1, in operation 110, the molecular dynamics simulation method may set the precision of the first data used for molecular dynamics simulation to be a first precision and may set the precision of the second data used for molecular dynamics simulation to be a second precision that is different from the first precision. In an example, the second precision may be higher (e.g., more precise) than the first precision.

In an example, the precision of the first data used for molecular dynamics simulation and the precision of the second data used for molecular dynamics simulation may be implemented in hardware (e.g., hardware implementing software and/or firmware). For example, data precision setting on the first data used for molecular dynamics simulation and data precision setting on the second data used for molecular dynamics simulation may be conducted by a configuration precision setting module of a molecular dynamics simulation apparatus.

For example, in an example herein, the configuration precision setting module may set data used for molecular dynamics simulation with different precisions and may set the precision of the first data used for molecular dynamics simulation to be the first precision and the precision of the second data used for molecular dynamics simulation to be the second precision that is higher than the first precision.

According to one or more embodiments, the molecular dynamics simulation method may set data used for molecular dynamics simulation with different precisions, e.g., may set the precision of the first data used for molecular dynamics simulation to be the first precision and the precision of the second data used for molecular dynamics simulation to be the second precision that is higher than the first precision. Through this, the molecular dynamics simulation method of one or more embodiments may advantageously reduce a computational amount used to conduct a molecular dynamics simulation by solving a problem of a vast computational amount caused by the use of the same precision (e.g., high precision) for the data used for the molecular dynamics simulation.

In one or more embodiments, the first precision may be single-precision and the second precision may be double-precision. For example, the first data used for molecular dynamics simulation may be a single-precision floating point and the second data used for molecular dynamics simulation may be a double-precision floating point. However, the first and second precisions of the present disclosure are not limited thereto, and the first and second precisions may be different precisions satisfying a condition that the second precision is higher than the first precision.

In one or more embodiments, the first data may include at least one of atom position coordinates and force coordinates and the second data may include at least one of atom velocity coordinates and a potential energy parameter.

The molecular dynamics simulation method of one or more embodiments may improve computational efficiency with a relatively small position coordinate error of an atom in the molecular dynamics simulation by setting at least one data precision of the force coordinates and position coordinates of an atom to be the first precision and setting at least one data precision of the potential energy parameter and velocity coordinates of an atom to be the second precision that is higher than the first precision. For example, the velocity coordinates of each atom may be configured as the second precision that is higher than the first precision such that an error in the position coordinates of the atom is not too large. Since the velocity coordinates may be sensitive to the position coordinates of the atom, a position coordinate error of each atom may be minimized as much as possible using the double precision.

For example, in one or more embodiments, as a result of analyzing to prevent affecting an error of position coordinates of the atom (also referred to as atom position coordinates), an atom position coordinate error may be directly affected by the velocity coordinates of the atom (also referred to as the atom velocity coordinates) and may not be affected by the force coordinates of the atom (also referred to as the atom force coordinates). Accordingly, the molecular dynamics simulation method of one or more embodiments may process at least one of the atom position coordinates and the force coordinates by using the first precision and may use the second precision to preserve at least one of the atom velocity coordinates and the potential energy parameter. Through such a combination of mixed precisions, the molecular dynamics simulation method of one or more embodiments may improve communication and computational efficiency while guaranteeing a small error (e.g., less than 1%) of the atom position coordinates.

In addition, in operation 120, the molecular dynamics simulation method may conduct the molecular dynamics simulation based on the first data having the first precision and the second data having the second precision.

As described above, when conducting the molecular dynamics simulation based on the first data and the second data, the molecular dynamics simulation method of one or more embodiments may prevent a problem of a vast computational amount caused by a typical molecular dynamics simulation method using data with the same precision (high precision, etc.) to conduct the molecular dynamics simulation.

In an example, the execution of the molecular dynamics simulation may be implemented by hardware (e.g., hardware implementing software and/or firmware). For example, the molecular dynamics simulation may be conducted by hardware implementing a mixed precision simulator.

In addition, when an iteration count of updates reaches a predetermined number or a time for conducting the molecular dynamics simulation reaches a predetermined length of time, the molecular dynamics simulation method may stop the execution of the molecular dynamics simulation and may output at least one of a simulation time, simulation precision, final position coordinates of the atom, final force coordinates, and final velocity coordinates.

The molecular dynamics simulation method of one or more embodiments may dramatically improve the computation speed of a molecular dynamics simulation experiment, may greatly decrease a limitation in the size of a storage space of a simulation platform, and may be compatible with a backend platform, such as a central processing unit (CPU) and a graphics processing unit (GPU). Thus, the molecular dynamics simulation method of one or more embodiments may improve the applicability of simulation for various apparatuses having limited storage spaces and may have wide applicability in smaller hardware terminals.

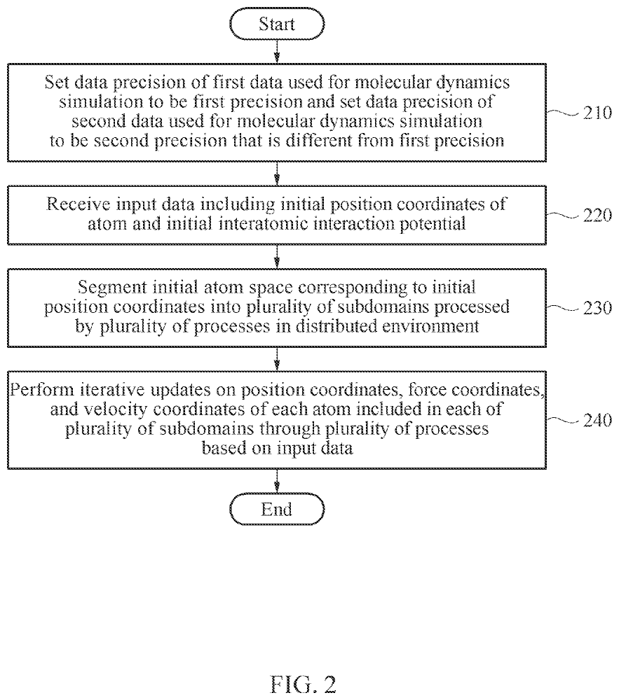

Hereinafter, a detailed description of FIG. 2 further describing an example of operation 120 is provided by combining various embodiments.

FIG. 2 illustrates an example of a molecular dynamics simulation method according to one or more embodiments.

Referring to FIG. 2, in operation 210, a molecular dynamics simulation method may set the precision of the first data used for molecular dynamics simulation to be a first precision and may set the precision of the second data used for molecular dynamics simulation to be a second precision that is different from the first precision. In an example, the second precision may be higher than the first precision.

In addition, in operation 220, the molecular dynamics simulation method may receive input data for the molecular dynamics simulation. In an example, the input data may include the initial position coordinates of an atom and an initial interatomic interaction potential.

The input data may be received by an initialization simulator. The initialization simulator may be implemented by hardware (e.g., hardware implementing software and/or firmware). The initialization simulator may obtain the initial position coordinates and the initial interatomic interaction potential based on the input data and may set an initialization parameter for the simulation. The initialization simulator may set a simulation command and may initialize the initial atom interaction potential for each atom, e.g., based on the initialization parameter.

In addition, the initialization simulator may set a range of a simulation space based on the input data. The initialization simulator may calculate the initial force coordinates of each atom based on the initialized initial interatomic interaction potential and may calculate the initial velocity coordinates of the atom using the initial force coordinates.

In operation 230, the initialization simulator may segment an initial atom space corresponding to the initial position coordinates into a plurality of subdomains respectively processed by a plurality of processes in a distributed environment.

In one or more embodiments, the initialization simulator may divide a total atom space into atom subdomains by a cube based on the number of simulated processes.

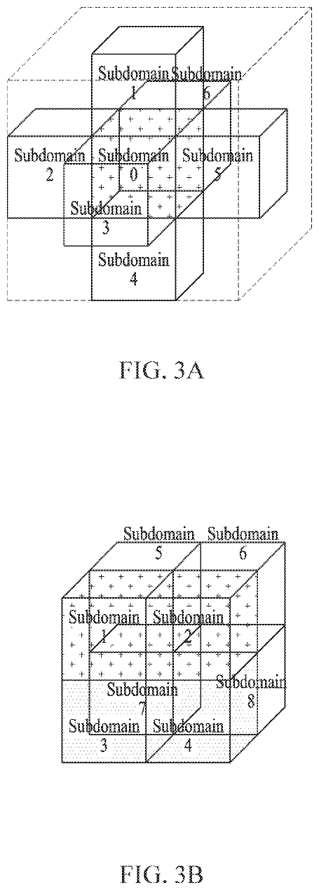

FIGS. 3A and 3B illustrate examples of a subdomain according to one or more embodiments.

Referring to FIG. 3A, each subdomain may have 6 neighbor subdomains (for example, an upper neighboring domain (a subdomain 1), a lower neighboring domain (a subdomain 4), a left neighboring domain (a subdomain 2), a right neighboring domain (a subdomain 5), a front neighboring domain (a subdomain 6), and a rear neighboring domain (a subdomain 6)). The initialization simulator may bind each process to each subdomain. For example, each process may calculate (for example, iteratively update) the position coordinates, force coordinates, and velocity coordinates of an atom in the subdomain. When the subdomains are divided by a cube, the number of subdomains is a power of 2. Accordingly, the number of processes may be a power of 2. For example, in one or more embodiments, the subdomains may be segmented in order from top to bottom, from left to right, and from front to back.

As shown in FIG. 3B, when 8 processes exist, the total atom space may be segmented into 8 cubic subdomain spaces (for example, the subdomains 1 to 8). One process may correspond to one subdomain.

In operation 240, a mixed precision simulator may iteratively update the position coordinates, the force coordinates, and the velocity coordinates of each atom in each subdomain through the plurality of processes based on the input data.

For example, according to one or more embodiments, the plurality of processes may process atomic data of a plurality of segmented subdomains in the total atom space in the distributed environment. Accordingly, the molecular dynamics simulation method of one or more embodiments may implement highly efficient parallel computing for the molecular dynamics simulation and this is particularly advantageous when the number of atoms is particularly great.

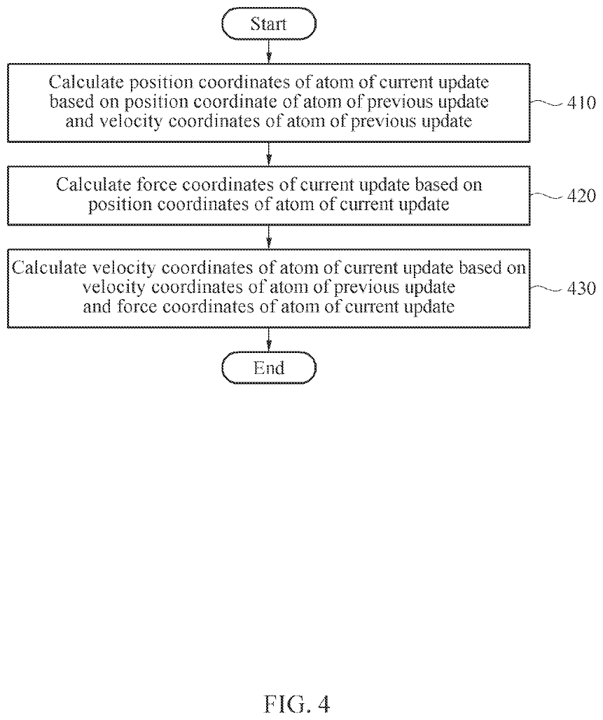

FIG. 4 illustrates an example of a portion of a process of performing an iterative update according to one or more embodiments.

In an example, the iterative update may represent a simulation round.

Referring to FIG. 4, in operation 410, a mixed precision simulator may calculate the position coordinates of an atom of a current update based on the position coordinates of the atom of a previous update and the velocity coordinates of the atom of a previous update.

The following describes operations of the mixed precision simulator from a process aspect. Each process P₁ may calculate, in parallel, position coordinates X of all atoms in each subdomain A₁. For example, based on velocity coordinates Vₛₜₑₚ₋₁ of each atom, position coordinates Xₛₜₑₚ may be newly calculated by Equation 1 shown below, for example.

Xₛₜₑₚ=Xₛₜₑₚ₋₁+Δᵥ×Vₛₜₑₚ₋₁ Equation 1:

In an example, the step may be an iteration count of updates (for example, an iteration count of simulations), Δᵥ may be predetermined by a velocity increment coefficient, Vₛₜₑₚ₋₁ may be velocity coordinates of an atom at a previous update (e.g., a step-1-th iterative update), Xₛₜₑₚ₋₁ may be position coordinates of the atom at the previous update, and Xₛₜₑₚ may be position coordinates of the atom at a current update (e.g., a step-th iterative update).

In addition, the position coordinates of the atom corresponding to a first update may be initial position coordinates, and the velocity coordinates of the atom corresponding to the first update may be initial velocity coordinates calculated through an initial interatomic interaction potential. In an example, the first update may be an update of which the step is 1 among iterative updates.

In operation 420, the mixed precision simulator may calculate the force coordinates of the atom of the current update based on the position coordinates of the atom of the current update.

In one or more embodiments, the mixed precision simulator may calculate an interatomic interaction potential based on the position coordinates of the atom of the current update. In one or more embodiments, the interatomic interaction potential may be a Tersoff interaction potential. However, the example described above is only an example, and the interatomic interaction potential of the present disclosure is not limited thereto and may be a different potential.

In operation 420, the mixed precision simulator may calculate the force coordinates of the atom of the current update based on the interatomic interaction potential. For example, for each atom, the mixed precision simulator may calculate a plurality of force coordinates applied to each atom by a different atom based on the interatomic interaction potential. In an example, the precision of the plurality of force coordinates may be the first precision. Furthermore, the mixed precision simulator may calculate the sum force coordinates of the plurality of force coordinates and may convert the sum force coordinates into the force coordinates of the atom of the current update. In an example, the precision of the sum force coordinates may be the second precision and the force coordinates of the atom of the current update may be the first precision.

For example, when calculating force coordinates between two atoms, the mixed precision simulator may obtain force coordinates of the first precision. However, when calculating the sum force coordinates corresponding to the plurality of force coordinates of a single atom, the mixed precision simulator may obtain the sum force coordinates and the second precision that is higher than the first precision. Accordingly, the molecular dynamics simulation method of one or more embodiments may reduce the computational workload while guaranteeing the calculation precision.

In one or more embodiments, each process may calculate new force coordinates of each atom based on the interatomic interaction potential using a single-precision floating point and may accumulate a total force aggregation using a double-precision floating point. For example, the new force coordinates of each atom receiving a force of a different atom may be represented by a single-precision floating point and the sum force coordinates of a sum force of each atom receiving a force of a different atom may be represented by a double-precision floating point. Accordingly, the molecular dynamics simulation method of one or more embodiments may reduce the computational workload while guaranteeing the calculation accuracy.

In operation 430, the mixed precision simulator may calculate the velocity coordinates of the atom of the current iterative update based on the velocity coordinates of the atom of the previous iterative update and the force coordinates of the atom of the current iterative update.

For example, each process P₁ may parallelly calculate the velocity coordinates V of all atoms in each subdomain A₁. For example, new velocity coordinates V may be calculated based on force coordinates F of each atom through Equation 2 shown below, for example.

Vₛₜₑₚ=Vₛₜₑₚ₋₁+Δ_{f}×Fₛₜₑₚ Equation 2:

In an example, the step may be an iteration count of updates (for example, an iteration count of simulations), Δ_{f} may be predetermined by a force increment coefficient, Vₛₜₑₚ₋₁ may be velocity coordinates of an atom of a previous update (e.g., a step-1-th iterative update), Vₛₜₑₚ may be velocity coordinates of the atom of a current update, and Fₛₜₑₚ may be force coordinates of the atom at the current update (e.g., a step-th iterative update).

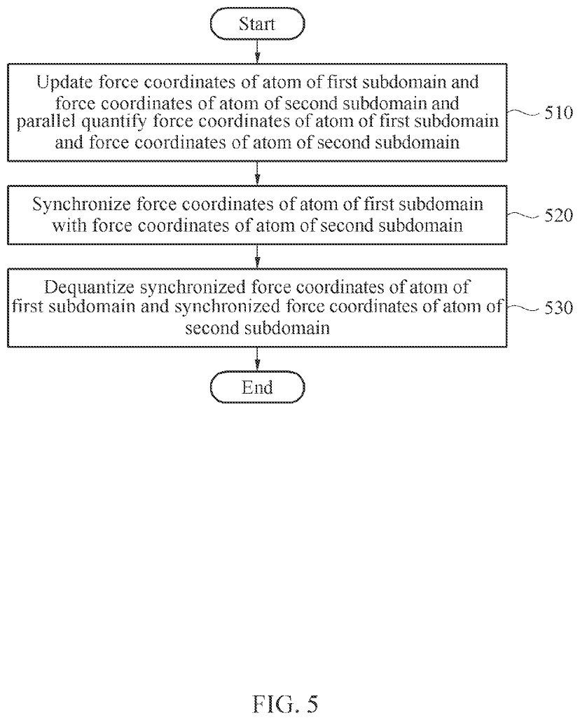

FIG. 5 illustrates an example of a portion of a process of performing an iterative update according to one or more embodiments.

For ease of description, a method of performing iterative updates in an example of a plurality of subdomains including a first domain and a second domain that is adjacent to the first subdomain is provided. The number of second subdomains may be one or more. In an example, a plurality of processes may include a first process corresponding to the first subdomain and a second process corresponding to the second subdomain.

Each process and neighboring processes in 6 directions (e.g., up, down, left, right, front, and rear) may guarantee that atom force coordinates in each process are the latest data by synchronizing updated atom force coordinates during each simulation round (for example, each iterative update).

Referring to FIG. 5, in operation 510, a mixed precision simulator may update the force coordinates of an atom of a first subdomain and the force coordinates of an atom of a second subdomain and may quantify, in parallel, the force coordinates of the atom of the first subdomain and the force coordinates of the atom of the second subdomain.

In an example, updates of the force coordinates of the atom of the first subdomain and the force coordinates of the atom of the second subdomain may represent force coordinates after force coordinates of the atom in an iterative update calculation calculated in operation 420.

By quantization of the force coordinates of the atom, the molecular dynamics simulation method of one or more embodiments may improve communication performance of a neighboring process by synchronizing coordinate data in the following operation. In addition, since the force coordinates of the atom may indirectly affect the velocity coordinates of the atom and the velocity coordinates of the atom may directly affect the position coordinates, the quantized force coordinates of the atom may barely affect an error of the position coordinates, and thus, may not significantly affect a simulation result.

In one or more embodiments, research on a parallel quantization method may provide multi-thread parallel computing capability by integrating GPUs and may implement a method of rapidly quantifying the first precision (e.g., a single-precision floating point type) to a lower precision (e.g., a short integer type).

In operation 520, the mixed precision simulator may synchronize force coordinates of parallel quantization of a first subdomain atom of a first process with force coordinates of parallel quantization of a second subdomain atom of a second process.

In one or more embodiments, synchronization of atom position coordinates data between two arbitrary neighboring processes may be implemented by using an open source message passing interface (Open MPI). However, the embodiment is only an example and is not limited thereto, and the synchronization may be implemented by using other synchronization methods or interfaces.

In operation 530, the mixed precision simulator may dequantize the synchronized force coordinates of the first subdomain atom and the synchronized force coordinates of the second subdomain atom.

In an example, when the dequantization method is the reverse of the quantization method, the force coordinates determined by dequantization may have the first precision, again.

Due to the high communication cost between processes, a data compression method based on parallel quantization may be adopted in data communication. The molecular dynamics simulation method of one or more embodiments may significantly reduce a duration and memory overhead of data communication by efficiently transmitting and receiving data using the principle of quantization and dequantization.

In addition, when force data of each atom between neighboring processes is to be synchronized, when performing distributed simulation in a multi-GPU environment, communication between processes may be completed through OpenMPI, and this may cause a huge amount of time for data synchronization and may affect the total simulation speed. Therefore, the molecular dynamics simulation method of one or more embodiments may improve the simulation speed by decreasing the volume of data in data synchronization between processes and accelerate the efficiency in data synchronization by configuring the force coordinates of each atom as the first precision (e.g., a single-precision floating point).

In addition, the molecular dynamics simulation method of one or more embodiments may adopt a parallel quantization method in a communication process of a parallel molecular dynamics simulation experiment, may compress and transmit force coordinate information in communication, and may decompress, calculate, and update the force coordinate information through parallel dequantization in response to receiving the information. Thus, the molecular dynamics simulation method of one or more embodiments may minimize an influence on the simulation accuracy while further improving the simulation speed. Accordingly, in a molecular dynamics simulation experiment with a vast volume of data, the molecular dynamics simulation method of one or more embodiments may significantly improve the simulation speed.

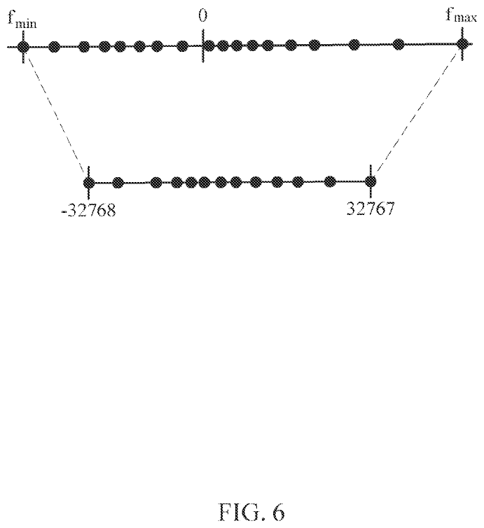

Hereinafter, a parallel quantization method according to one or more embodiments is described with reference to FIGS. 6 to 8.

FIG. 6 illustrates an example of a parallel quantization method according to one or more embodiments.

Referring to FIG. 6, as a parallel quantization method, illustrated is an example of a min-max quantization method. However, the above embodiment is only an example and is not limited thereto, and the parallel quantization method may be another quantization method. The mix-max quantization method may be expressed by Equations 3 to 5 shown below, for example.

$$scale = \frac{{f}_{max} - {f}_{min}}{{Q}_{max} - {Q}_{min}} \qquad Equation 3$$

$$zero = {Q}_{max} - \frac{{f}_{max}}{scale} \qquad Equation 4$$

$$Q = \frac{f}{scale} + zero \qquad Equation 5$$

In an example, when force coordinates are quantified as 16-byte integers, Qₘₐₓ may be 32767 and Qₘᵢₙ may be −32768.

In Equations 3 to 5, scale may denote a quantization coefficient, zero may denote a quantization offset, f may denote first precision force coordinates (e.g., an original single-precision (flocat) floating point), fₘₐₓ may denote a maximum value of the first precision force coordinates, fₘᵢₙ may denote a minimum value of the first precision force coordinates, and Q may denote a quantized integer.

For example, the molecular dynamics simulation method may generate a cache area (e.g., a short integer cache area) having a length of N in a GPU memory based on the number of pieces of atomic coordinate data to be synchronized. The total cache area may be used to store quantized and compressed short integer data. For example, the molecular dynamics simulation method may assign N pieces of atomic coordinate data to N threads in a GPU, respectively, and may cause each thread to calculate first precision data (e.g., a single-precision floating point) and may quantize as one short integer-type number.

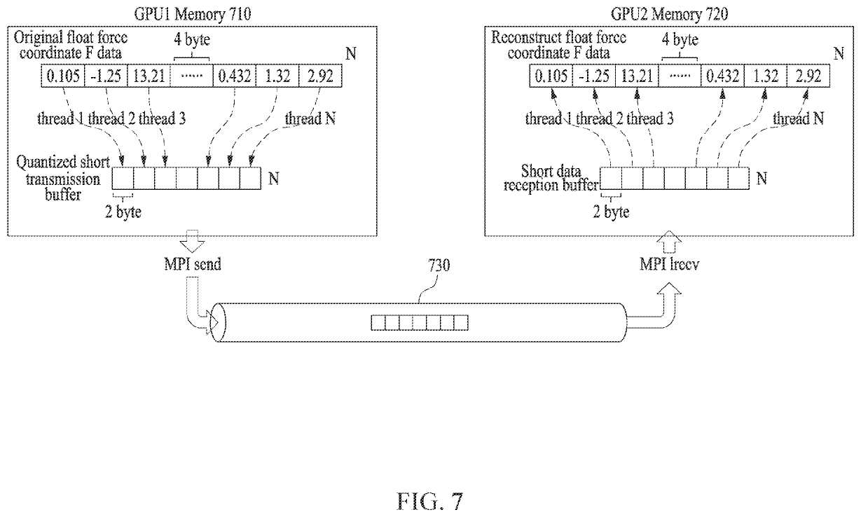

FIG. 7 illustrates an example of transmitting data through parallel quantization according to one or more embodiments.

Referring to FIG. 7, when all pieces of data to be transmitted by a graphics processor GPU1 have been quantified, data of a quantization buffer (for example, a GPU1 memory 710) using an interface MPI_send may be directly transmitted to a reception buffer memory (for example, a GPU2 memory 720) of a graphics processor GPU2 via a network 730. The GPU2 memory 720 may receive the quantified data through an interface MPI_Irecv.

When quantized data is received in GPU2, the quantized data may be reconstructed as first precision force coordinates by performing dequantization on the quantized data. In addition, a dequantization process may be completed in parallel by executing each piece of data to each thread of a GPU. The dequantization may be performed using Equation 6 shown below, for example, based on a parameter used for quantization.

f=(Q−zero)×scale Equation 6:

In an example, scale may denote a quantization coefficient, zero may denote a quantization offset, f may denote first precision force coordinates (e.g., an original single-precision (flocat) floating point), and Q may be a quantized integer.

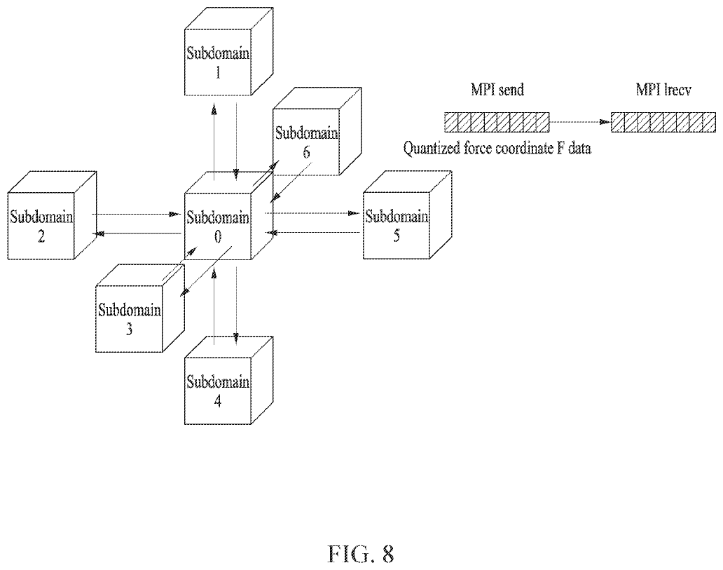

FIG. 8 illustrates an example of parallel quantization between neighboring processes according to one or more embodiments.

Referring to FIG. 8, a neighboring process may update only atomic coordinates in their own subdomain and then may synchronize and quantify quantized atomic coordinates using an MPI interface. The molecular dynamics simulation method of one or more embodiments may increase the synchronization efficiency of atomic coordinates between neighboring processes by reducing the size of synchronized data by greatly compressing synchronized force coordinates through a parallel quantization method.

In FIG. 8, each process may synchronize data with neighboring 6 processes, in up, down, left, right, front, and rear. For example, data of a subdomain 0 may be synchronized with data of neighboring subdomains 1 to 6. A transmission process may transmit the data of a buffer using an interface MPI_send and a reception process may receive data by a buffer using an interface MPI_Irecv.

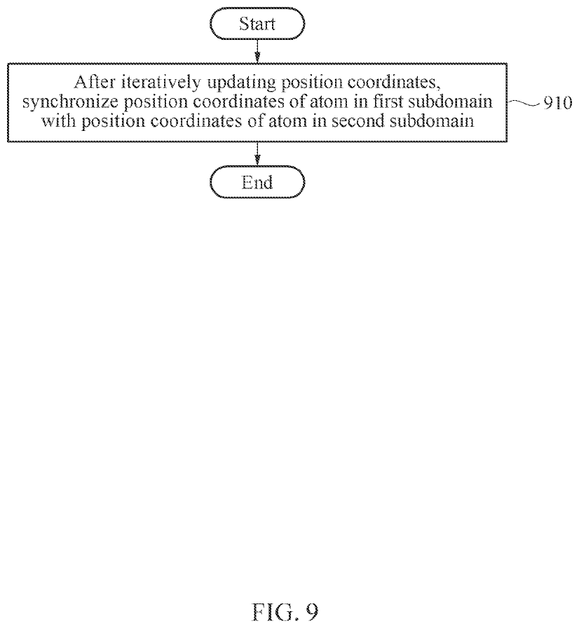

FIG. 9 illustrates an example of a portion of a process of performing an iterative update according to one or more embodiments.

For ease of description, a method of performing iterative updates in an example of a plurality of subdomains including a first domain and a second domain that is adjacent to the first subdomain.

Referring to FIG. 9, in operation 910, in a molecular dynamics simulation method, when position coordinates have been iteratively updated, a mixed precision simulator may synchronize the position coordinates of an atom in a first subdomain with position coordinates of an atom in a second subdomain.

In an example, the time point after each iterative update of position coordinates may represent a time point after calculating the position coordinates of an atom in a calculation iterative update in operation 410.

In addition, when force data of each atom between neighboring processes is to be synchronized, when performing distributed simulation in a multi-GPU environment, communication between processes may be completed through OpenMPI, and this may cause a huge amount of time for data synchronization and may affect the total simulation speed. Therefore, the molecular dynamics simulation method of one or more embodiments may improve the simulation speed by decreasing the volume of synchronized data between processes and accelerate the efficiency in data synchronization by configuring the coordinates of each atom as the first precision (e.g., a single-precision floating point).

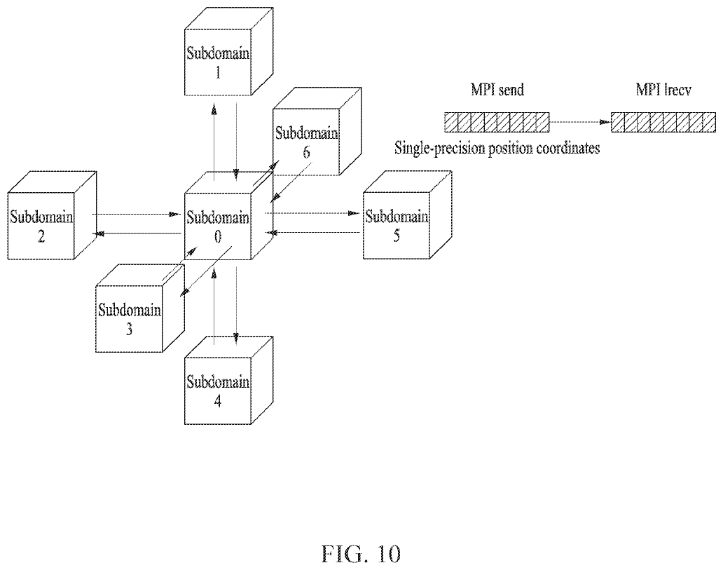

FIG. 10 illustrates an example of synchronizing position coordinates between neighboring processes according to one or more embodiments.

Referring to FIG. 10, each process may update only atom position coordinates in their own subdomain and then may synchronize atom position coordinate data using OpenMPI. For example, data of a subdomain 0 may be synchronized with data of neighboring subdomains 1 to 6.

According to one or more embodiments, a molecular dynamics simulation method may reduce the size of synchronization data and the volume of communication by setting atom coordinate data to be data (e.g., a single-precision floating point) of a first precision. When synchronizing the atom position coordinates, the first precision force coordinates may be transmitted using an interface MPI_send and may receive the first precision force coordinates using an interface MPI_Irecv.

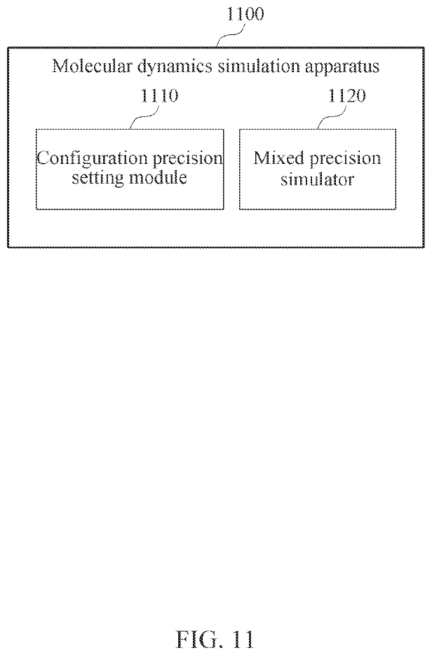

FIG. 11 illustrates an example of a schematic configuration of a molecular dynamics simulation apparatus according to one or more embodiments.

Referring to FIG. 11, a molecular dynamics simulation apparatus 1100 may include a configuration precision setting module 1110 and a mixed precision simulator 1120.

The configuration precision setting module 1110 may set the precision of the first data used for molecular dynamics simulation and the precision of the second data used for molecular dynamics simulation to be first precision and second precision, respectively. In an example, the second precision may be higher than the first precision.

The mixed precision simulator 1120 may perform molecular dynamics simulation based on the first data of the first precision and the second data of the second precision.

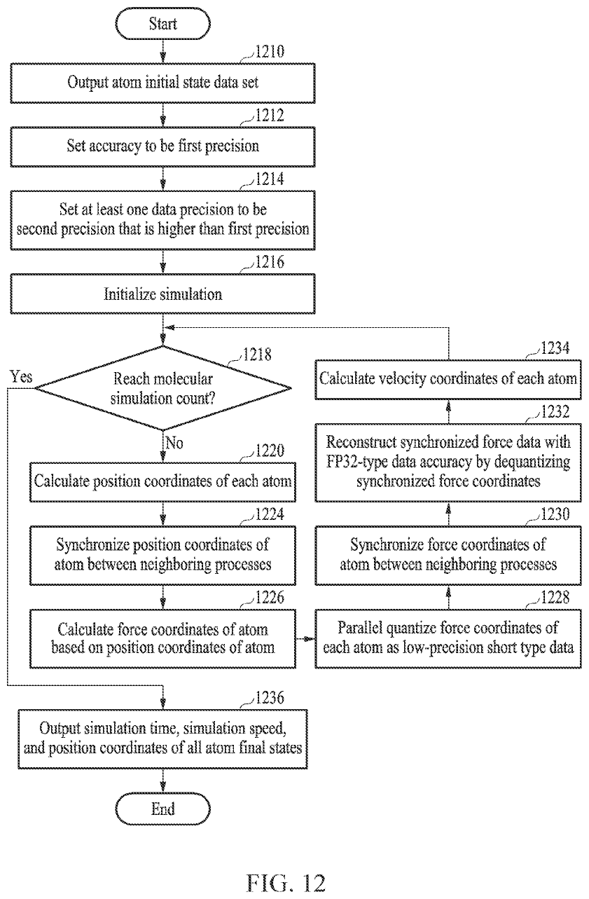

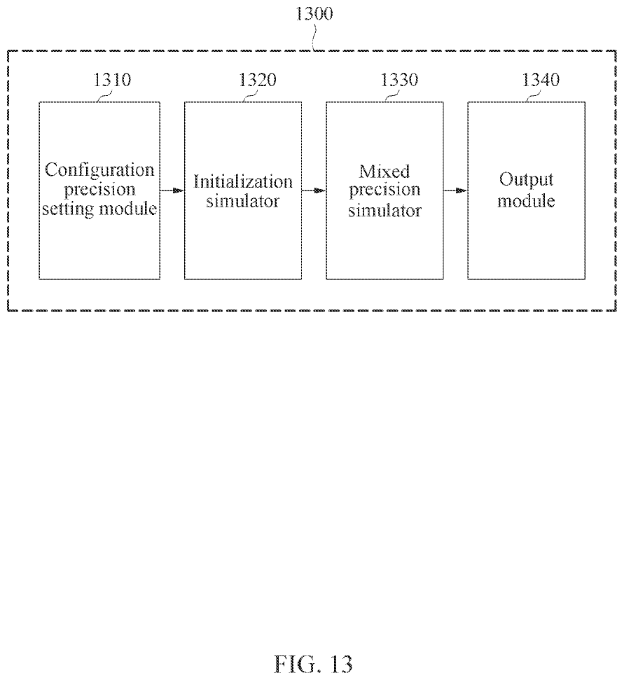

In addition, selectively, the molecular dynamics simulator 1100 may further include an initialization simulator and an output module. An embodiment including the initialization simulator and the output module is described with reference to FIGS. 12 and 13.

FIG. 12 illustrates an example of a detailed process of a molecular dynamics simulation method according to one or more embodiments.

FIG. 13 illustrates an example of a configuration of a molecular dynamics simulation apparatus according to one or more embodiments.

Hereinabove, each operation of the molecular dynamics simulation method according to one or more embodiments is described with reference to FIGS. 1 to 11. In an example, the molecular dynamics simulation method according to one or more embodiments is briefly described.

Referring to FIGS. 12 and 13, a molecular dynamics simulation apparatus 1300 may include a configuration precision setting module 1310, an initialization simulator 1320, a mixed precision simulator 1330, and an output module 1340.

In operation 1210, the configuration precision setting module 1310 may output an atom initial state data set based on input data. The atom initial state data set may be used to set an initial state of an atom.

In addition, in operation 1212, the configuration precision setting module 1310 may set data accuracy of at least one of position coordinates and force coordinates of the atom to be first precision. As a non-limiting example, the configuration precision setting module 1310 may set a data type of position coordinates and force coordinates of each atom to be first precision (e.g., a single-precision FP32).

In operation 1214, the configuration precision setting module 1310 may set data precision of at least one of a potential energy parameter and velocity coordinates of the atom to be a second precision that is higher than the first precision. As a non-limiting example, the configuration precision setting module 1310 may set a data type of a potential energy parameter and force coordinates of each atom to be second precision (e.g., a double-precision FP64).

In operation 1216, the initialization simulator 1320 may initialize simulation based on the data set by the configuration precision setting module 1310. As a non-limiting example, the initialization simulator 1320 may read the initial size of a simulation space and the initial position coordinates of an atom and may assign an initialization potential to the atom.

In operation 1218, the mixed precision simulator 1330 may determine whether a simulation count reaches a molecular dynamics simulation count.

In operation 1220, as a result of operation 1218, when it is determined that the molecular dynamics simulation count is not reached, the mixed precision simulator 1330 may calculate the position coordinates of each atom.

In addition, in operation 1224, the mixed precision simulator 1330 may synchronize position coordinates of the atom between neighboring processes.

In addition, in operation 1226, the mixed precision simulator 1330 may calculate the force coordinates of the atom based on the position coordinates of the atom.

In addition, in operation 1228, the mixed precision simulator 1330 may parallelly quantize force coordinates of each atom as low-precision short type data.

In addition, in operation 1230, the mixed precision simulator 1330 may synchronize the force coordinates of the atom between neighboring processes.

In addition, in operation 1232, the mixed precision simulator 1330 may reconstruct synchronized force data with FP32-type data accuracy by dequantizing the synchronized force coordinates.

In addition, in operation 1234, the mixed precision simulator 1330 may calculate the velocity coordinates of each atom. When the velocity coordinates of each atom are calculated, the mixed precision simulator 1330 may return to operation 1218 and may determine again whether the simulation count reaches the molecular dynamics simulation count.

In addition, in operation 1236, as a result of operation 1218, when it is determined that the molecular dynamics simulation count is reached, the output module 1340 may output a simulation time, a simulation speed, and position coordinates of all atomic final states.

Referring to FIG. 13, detailed descriptions of each component of the molecular dynamics simulation apparatus 1390 are provided.

Firstly, the configuration precision setting module 1310 may configure the precision of data used for the molecular dynamics simulation. As a non-limiting example, the configuration precision setting module 1310 may improve the total simulation speed by setting the atom position coordinates and the atom force coordinates to be single-precision and setting the atom velocity coordinates and the potential energy parameter to be double-precision.

The initialization simulator 1320 may initialize the simulation based on the atom initial position coordinates and a simulation space coordinate range. For example, the initialization simulator 1320 may read the initial position coordinates of the atom and simulation space coordinates, may initialize the simulation parameter, may set a simulation name, and may assign one initialized potential to each atom.

When the simulation is initialized, the mixed precision simulator 1330 may begin an iterative update of atom data. In each update of iterative updates, the mixed precision simulator 1330 may calculate the position coordinates of each atom through the following six steps.

The mixed precision simulator 1330 may calculate the position coordinates of each atom in a first step. As a non-limiting example, the mixed precision simulator 1330 may update the initialization velocity coordinates of each atom based on an initialized interaction potential to each atom and may calculate the position coordinates of each atom based on the initialization velocity coordinates of each atom.

Then, the mixed precision simulator 1330 may synchronize the coordinates of each atom between neighboring processes in a second step. As a non-limiting example, each process and neighboring processes thereof in six directions, up, down, left, right, front, and rear, may synchronously update atom position coordinates. Synchronization of atom position coordinate data between two neighboring processes may be implemented by using an OpenMPI interface. Because the position coordinate data of each atom may be flexibly configured as a single-precision or double-precision floating point type, the volume of synchronization data may decrease by setting the position coordinate data of each atom to be a single-precision floating point type.

In addition, the mixed precision simulator 1330 may calculate the force coordinates of each atom in a third step.

In addition, the mixed precision simulator 1330 may parallelly quantize the force coordinates of each atom in a fourth step. For example, the mixed precision simulator 1330 may further compress the force coordinates of each atom by processing an advanced and parallelized job using a multi-thread characteristic of a GPU.

In addition, the mixed precision simulator 1330 may synchronize the force coordinates of the atom between neighboring processes in a fifth step. For example, the mixed precision simulator 1330 may cause each process and neighboring processes thereof in six directions, up, down, left, right, front, and rear, to synchronize the compressed force coordinates of the atom.

In addition, the mixed precision simulator 1330 may calculate the velocity coordinates of each atom in a sixth step. For example, each process may calculate new velocity coordinates for each atom based on the latest atom force coordinates.

When the simulation is completed, the output module 1340 may output a simulation result. For example, the simulation result may include a simulation time, speed, and a final atom coordinate position.

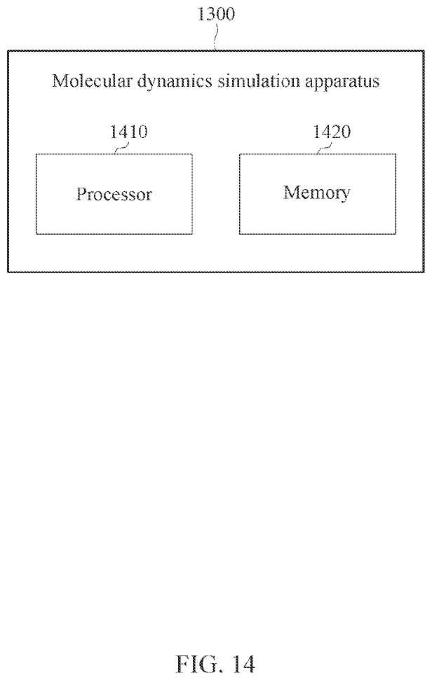

FIG. 14 illustrates an example of a molecular dynamics simulation system according to one or more embodiments.

Referring to FIG. 14, a molecular dynamics simulation apparatus 1400 according to one or more embodiments may include a processor 1410 (e.g., one or more processors) and a memory 1420 (e.g., one or more memories). In an example, the memory 1420 may store a computer program. In an example, when the computer program is executed by the processor 1410, the computer program may implement at least one method described with reference to FIGS. 1 to 13. Because the description is provided above, a description of the at least one method that is executed by the processor 1410 and is described with reference to FIGS. 1 to 13 is omitted. For example, the memory 1420 may be or include a non-transitory computer-readable storage medium storing instructions that, when executed by the processors 1410, configure the processor 1410 to perform any one, any combination, or all of the operations and methods described herein with reference to FIGS. 1-13.

The GPU1 memory 710. GPU2 memory 720, molecular dynamics simulation apparatus 1100, configuration precision setting module 1110, mixed precision simulator 1120, molecular dynamics simulation apparatus 1300, configuration precision setting module 1310, initialization simulator 1320, mixed precision simulator 1330, output module 1340, processor 1410, memory 1420, and other apparatuses, devices, units, modules, and components disclosed and described herein with respect to FIGS. 1-14 are implemented by or representative of hardware components. As described above, or in addition to the descriptions above, examples of hardware components that may be used to perform the operations described in this application where appropriate include controllers, sensors, generators, drivers, memories, comparators, arithmetic logic units, adders, subtractors, multipliers, dividers, integrators, and any other electronic components configured to perform the operations described in this application. In other examples, one or more of the hardware components that perform the operations described in this application are implemented by computing hardware, for example, by one or more processors or computers. A processor or computer may be implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices that is configured to respond to and execute instructions in a defined manner to achieve a desired result. In one example, a processor or computer includes, or is connected to, one or more memories storing instructions or software that are executed by the processor or computer. Hardware components implemented by a processor or computer may execute instructions or software, such as an operating system (OS) and one or more software applications that run on the OS, to perform the operations described in this application. The hardware components may also access, manipulate, process, create, and store data in response to execution of the instructions or software. For simplicity, the singular term “processor” or “computer” may be used in the description of the examples described in this application, but in other examples multiple processors or computers may be used, or a processor or computer may include multiple processing elements, or multiple types of processing elements, or both. For example, a single hardware component or two or more hardware components may be implemented by a single processor, or two or more processors, or a processor and a controller. One or more hardware components may be implemented by one or more processors, or a processor and a controller, and one or more other hardware components may be implemented by one or more other processors, or another processor and another controller. One or more processors, or a processor and a controller, may implement a single hardware component, or two or more hardware components. As described above, or in addition to the descriptions above, example hardware components may have any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in FIGS. 1-14 that perform the operations described in this application are performed by computing hardware, for example, by one or more processors or computers, implemented as described above implementing instructions or software to perform the operations described in this application that are performed by the methods. For example, a single operation or two or more operations may be performed by a single processor, or two or more processors, or a processor and a controller. One or more operations may be performed by one or more processors, or a processor and a controller, and one or more other operations may be performed by one or more other processors, or another processor and another controller. One or more processors, or a processor and a controller, may perform a single operation, or two or more operations.

Instructions or software to control computing hardware, for example, one or more processors or computers, to implement the hardware components and perform the methods as described above may be written as computer programs, code segments, instructions or any combination thereof, for individually or collectively instructing or configuring the one or more processors or computers to operate as a machine or special-purpose computer to perform the operations that are performed by the hardware components and the methods as described above. In one example, the instructions or software include machine code that is directly executed by the one or more processors or computers, such as machine code produced by a compiler. In another example, the instructions or software includes higher-level code that is executed by the one or more processors or computer using an interpreter. The instructions or software may be written using any programming language based on the block diagrams and the flow charts illustrated in the drawings and the corresponding descriptions herein, which disclose algorithms for performing the operations that are performed by the hardware components and the methods as described above.

The instructions or software to control computing hardware, for example, one or more processors or computers, to implement the hardware components and perform the methods as described above, and any associated data, data files, and data structures, may be recorded, stored, or fixed in or on one or more non-transitory computer-readable storage media, and thus, not a signal per se. As described above, or in addition to the descriptions above, examples of a non-transitory computer-readable storage medium include one or more of any of read-only memory (ROM), random-access programmable read only memory (PROM), electrically erasable programmable read-only memory (EEPROM), random-access memory (RAM), dynamic random access memory (DRAM), static random access memory (SRAM), flash memory, non-volatile memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, blue-ray or optical disk storage, hard disk drive (HDD), solid state drive (SSD), flash memory, a card type memory such as multimedia card micro or a card (for example, secure digital (SD) or extreme digital (XD)), magnetic tapes, floppy disks, magneto-optical data storage devices, optical data storage devices, hard disks, solid-state disks, and any other device that is configured to store the instructions or software and any associated data, data files, and data structures in a non-transitory manner and provide the instructions or software and any associated data, data files, and data structures to one or more processors or computers so that the one or more processors or computers can execute the instructions. In one example, the instructions or software and any associated data, data files, and data structures are distributed over network-coupled computer systems so that the instructions and software and any associated data, data files, and data structures are stored, accessed, and executed in a distributed fashion by the one or more processors or computers.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents.

Therefore, in addition to the above and all drawing disclosures, the scope of the disclosure is also inclusive of the claims and their equivalents, i.e., all variations within the scope of the claims and their equivalents are to be construed as being included in the disclosure.

What is claimed is:

1. A processor-implemented method with molecular dynamics simulation, the method comprising:
setting a precision of first data used for a molecular dynamics simulation to be a first precision;
setting a precision of second data used for the molecular dynamics simulation to be a second precision that is different from the first precision; and
conducting the molecular dynamics simulation based on the first data of the first precision and the second data of the second precision.

2. The method of claim 1, wherein
the first data comprises either one or both of position coordinates and force coordinates of an atom,
the second data comprises either one or both of a potential energy parameter and velocity coordinates of the atom, and
the second precision comprises precision that is higher than the first precision.

3. The method of claim 1, further comprising:
receiving input data comprising initial position coordinates of an atom and an interatomic initial interaction potential; and
segmenting an initial atom space corresponding to the initial position coordinates into a plurality of subdomains processed by a plurality of processes in a distributed environment,
wherein the conducting of the molecular dynamics simulation comprises iteratively updating position coordinates, force coordinates, and velocity coordinates of each atom comprised in each of the plurality of subdomains through the plurality of processes based on the input data.

4. The method of claim 3, wherein
the plurality of subdomains comprises a first subdomain and a second subdomain neighboring to the first subdomain,
the plurality of processes comprises a first process corresponding to the first subdomain and a second process corresponding to the second subdomain, and
the iteratively updating comprises:
updating force coordinates of an atom of the first subdomain and force coordinates of an atom of the second subdomain;
parallelly quantify the force coordinates of the atom of the first subdomain and the force coordinates of the atom of the second subdomain;
synchronizing the force coordinates of the atom of the first subdomain with the force coordinates of the atom of the second subdomain; and
dequantizing synchronized force coordinates of the atom of the first subdomain and synchronized force coordinates of the atom of the second subdomain.

5. The method of claim 3, wherein the iteratively updating comprises iteratively updating each of the plurality of subdomains.

6. The method of claim 3, wherein the iteratively updating of each of the plurality of subdomains comprises:
determining position coordinates of the atom of a current update based on position coordinates of the atom of a previous update and velocity coordinates of the atom of the previous update;
determining force coordinates of the atom based on the position coordinates of the atom in the current update and the force coordinates of the atom of the current update; and
determining velocity coordinates of the atom of the current update based on velocity coordinates of the atom of a previous update and velocity coordinates of the atom of the previous update.

7. The method of claim 6, wherein
the determining of the position coordinates of the atom comprises determining position coordinates of the atom corresponding to a first update to be the initial position coordinates, and
the determining of the velocity coordinates of the atom of the current update comprises determining the velocity coordinates of the atom through the interatomic initial interaction potential, wherein the velocity coordinates of the atom corresponds to the first update.

8. The method of claim 6, wherein the determining of the force coordinates of the atom of the current update comprises:
determining the interatomic interaction potential based on the position coordinates of the atom of the current update; and
determining the force coordinates of the atom of the current update based on the interatomic interaction potential.

9. The method of claim 8, wherein, for each atom, the determining of the force coordinates of the atom of the current update based on the interatomic interaction potential comprises:
determining a plurality of force coordinates applied to the atom by another atom based on the interatomic interaction potential;
determining sum force coordinates of the plurality of force coordinates; and
converting the sum force coordinates into force coordinates of the atom of the current update.

10. The method of claim 9, wherein
precision of the plurality of force coordinates and precision of the force coordinates of the atom of the current update are the first precision,
precision of the sum force coordinates is the second precision, and
the second precision comprises precision that is higher than the first precision.

11. An apparatus with molecular dynamics simulation, the apparatus comprising:
one or more processors configured to:
set a precision of first data used for a molecular dynamics simulation to be first precision;
set a precision of second data used for the molecular dynamics simulation to be second precision that is different from the first precision; and
conduct the molecular dynamics simulation based on the first data of the first precision and the second data of the second precision.

12. The apparatus of claim 11, wherein
the first data comprises either one or both of position coordinates and force coordinates of an atom,
the second data comprises either one or both of a potential energy parameter and velocity coordinates of the atom, and
the second precision comprises precision that is higher than the first precision.

13. The apparatus of claim 11, wherein the one or more processors are configured to:
receive input data comprising initial position coordinates of an atom and an interatomic initial interaction potential;
segment an initial atom space corresponding to initial position coordinates into a plurality of subdomains processed by a plurality of processes in a distributed environment; and
for the conducting of the molecular dynamics simulation, iteratively update position coordinates, force coordinates, and velocity coordinates of each atom comprised in each of the plurality of subdomains through the plurality of processes based on the input data.

14. The apparatus of claim 13, wherein
the plurality of subdomains comprises a first subdomain and a second subdomain neighboring to the first subdomain,
the plurality of processes comprises a first process corresponding to the first subdomain and a second process corresponding to the second subdomain, and
for the iteratively updating, the one or more processors are configured to:
update the force coordinates of the atom of the first subdomain with the force coordinates of the atom of the second subdomain;
parallelly quantify the force coordinates of the atom of the first subdomain and the force coordinates of the atom of the second subdomain;
synchronize the force coordinates of the atom of the first subdomain with the force coordinates of the atom of the second subdomain; and
dequantize synchronized force coordinates of the atom of the first subdomain and synchronized force coordinates of the atom of the second subdomain.

15. The apparatus of claim 13, wherein, for the iteratively updating, the one or more processors are configured to iteratively update each of the plurality of subdomains.

16. The apparatus of claim 13, wherein, for the iteratively updating of each of the plurality of subdomains, the one or more processors are configured to:
determine position coordinates of the atom of a current update based on position coordinates of the atom of a previous update and velocity coordinates of the atom of the previous update;
determine force coordinates of the atom based on the position coordinates of the atom in the current update and the force coordinates of the atom of the current update; and
determine velocity coordinates of the atom of the current update based on velocity coordinates of the atom of a previous update and velocity coordinates of the atom of the previous update.

17. The apparatus of claim 16, wherein the one or more processors are configured to:
for the determining of the position coordinates of the atom, determine position coordinates of the atom corresponding to a first update to be the initial position coordinates, and
for the determining of the velocity coordinates of the atom of the current update, determine the velocity coordinates of the atom through the interatomic initial interaction potential, wherein the velocity coordinates of the atom corresponds to the first update.

18. The apparatus of claim 16, wherein, for the determining of the force coordinates of the atom of the current update, the one or more processors are configured to:
determine the interatomic interaction potential based on the position coordinates of the atom of the current update; and
determine the force coordinates of the atom of the current update based on the interatomic interaction potential.

19. The apparatus of claim 18, wherein, for the determining of the force coordinates of the atom of the current update based on the interatomic interaction potential, for each atom, the one or more processors are configured to:
determine a plurality of force coordinates applied to the atom by another atom based on the interatomic interaction potential;
determine sum force coordinates of the plurality of force coordinates; and
convert the sum force coordinates into force coordinates of the atom of the current update.

20. The apparatus of claim 19, wherein
precision of the plurality of force coordinates and precision of the force coordinates of the atom of the current update are the first precision,
precision of the sum force coordinates is the second precision, and
the second precision comprises precision that is higher than the first precision.
